(19) United States
(12) Patent Application Publication
(10) Pub. No.: US 2025/0258183 A1
(43) Pub. Date: Aug. 14, 2025

(54) ENZYME-LINKED IMMUNOSORBENT ASSAY (ELISA) FOR ANTI-BSEP AUTOANTIBODY

(71) Applicant: CHILDREN'S HOSPITAL MEDICAL CENTER, Cincinnati, OH (US)

(72) Inventors: Akihiro Asai, Cincinnati, OH (US); Hisamitsu Hayashi, Tokyo (JP)

(21) Appl. No.: 19/048,636

(22) Filed: Feb. 7, 2025

Related U.S. Application Data
(60) Provisional application No. 63/552,073, filed on Feb. 9, 2024.

(51) Int. Cl. G01N 33/68; C07K 14/47; G01N 33/543

(52) U.S. Cl. CPC G01N 33/6854; C07K 14/4702; G01N 33/543; G01N 2800/52

(57) ABSTRACT

Methods of detecting the presence and quantity of anti-BSEP antibodies in a biological sample from a subject include contacting the biological sample with one or more BSEP peptides to form an antibody-BSEP peptide complex, and detecting the amount of the antibody-BSEP peptide complex in the biological sample. The one or more BSEP peptides may include one or more of a full length BSEP peptide (SEQ ID NO:1), and/or one or more epitopes of BSEP.

### CROSS-REFERENCE TO RELATED APPLICATIONS

This application claims priority to and the benefit of U.S. Provisional Application Ser. No. 63/552,073, filed Feb. 9, 2024, the entire contents of which are incorporated herein by reference for all purposes.

### REFERENCE TO SEQUENCE LISTING

A Sequence Listing submitted as an ST.26 standards compliant XML file via Patent Center is hereby incorporated herein by reference. The file name of the XML file for the Sequence Listing is CHMC_2022-0708_SL.xml, the date of creation of the XML file is Feb. 2, 2025, and the size of the XML file is 9.27 kilobytes.

### BACKGROUND

Cholestasis in children has many different causes. Progressive familial intrahepatic cholestasis (PFIC)—also referred to as Byler's disease, Byler's syndrome, benign recurrent intrahepatic cholestasis or familial hypercholanemia—is an inherited disease that disrupts the genes encoding protein transporters responsible for bile metabolism. These mutant proteins result in the impairment of bile flow through the liver, leading to severe intrahepatic cholestasis and progressive chronic liver disease.

Progressive familial intrahepatic cholestasis type 2 (PFIC2)—one of many different subtypes of PFIC—is a rare hereditary disorder presenting early in life, with high serum bile salt and bilirubin levels. Signs and symptoms may include severe itching, jaundice, failure to thrive, portal hypertension (high blood pressure in the vein that provides blood to the liver) and hepatosplenomegaly (enlarged liver and spleen). Affected people also have an increased risk of developing hepatocellular carcinoma (a form of liver cancer). PFIC2 is caused by variants in the ABCB11 gene encoding the Bile Salt Export Pump (BSEP). The gene locus is on chromosome 2 (2q24) and is similarly inherited in an autosomal recessive fashion.

BSEP is a transporter protein that is expressed at the canalicular membrane of hepatocytes, and is the primary exporter of bile acids. BSEP malfunction leads to failure of bile salt secretion from hepatocytes into bile canaliculi and accumulation of bile inside the hepatocytes. This results in severe impaired bile flow and hepatocellular damage. On immunohistochemical staining, BSEP is usually not detectable in PFIC2, and if there is any protein present, it is usually non-functional.

Treatments for PFIC and PFC2 can vary, but ultimately, liver transplantation is needed in most patients because they often develop progressive liver fibrosis, cirrhosis and end stage liver disease. Following successful liver transplantation, an acquired form of intrahepatic cholestasis may develop in PFIC2 patients. Known as “alloimmunity,” antibodies against the liver graft of the donor in the patient cause complications, such as cholestasis and a return of PFIC2 symptoms. Approximately 8-33% of patients with PFIC2 who undergo liver transplant develop antibodies against the normal BSEP (anti-BSEP antibodies). Anti-BSEP antibodies present in the patient's serum can inhibit BSEP from the extracellular, biliary side. In addition to liver transplant recipients, recipients of gene therapy or messenger RNA therapy may also induce autoimmunity to the “corrected” BSEP after treatment, leading to a return of BSEP deficiency.

### SUMMARY

According to embodiments of the present disclosure, a method of detecting the presence and quantity of anti-BSEP antibodies in a biological sample from a subject includes contacting the biological sample with one or more BSEP peptides to form an antibody-BSEP peptide complex, and detecting the amount of the antibody-BSEP peptide complex in the biological sample. In some aspects, the BSEP peptide may be immobilized on a solid support, and the contacting may include introducing the biological sample to the BSEP peptide on the solid support. And in some aspects, the detecting the presence and quantity of the anti-BSEP antibodies may be carried out via enzyme-linked immunosorbent assay (ELISA).

In some aspects, the one or more BSEP peptides may include one or more of a full length BSEP peptide (SEQ ID NO:1), or one or more epitopes of BSEP. In some aspects, the one or more epitopes of BSEP may be selected from the peptide of SEQ ID NO: 2, one or more epitopes of the full length BSEP peptide having a length of 18-20 amino acids, and combinations thereof. For example, in some aspects, the one or more epitopes may be selected from the peptide of SEQ ID NO: 2, the peptide of SEQ ID NO: 3, the peptide of SEQ ID NO: 4, the peptide of SEQ ID NO: 5, the peptide of SEQ ID NO: 6, the peptide of SEQ ID NO: 7, the peptide of SEQ ID NO: 8, and combinations thereof.

According to some aspects, the one or more BSEP peptides may include a single BSEP peptide. For example, in some aspects, the single BSEP peptide may be selected from the full length BSEP peptide (SEQ ID NO: 1), the peptide of SEQ ID NO: 2, the peptide of SEQ ID NO: 3, the peptide of SEQ ID NO: 4, the peptide of SEQ ID NO: 5, the peptide of SEQ ID NO: 6, the peptide of SEQ ID NO: 7, or the peptide of SEQ ID NO: 8.

In some aspects, the one or more BSEP peptides may include a panel of 2 or more peptides. For example, in some aspects, the panel of 2 or more BSEP peptides may include one of: 1) the full length BSEP peptide (SEQ ID NO: 1), and the peptide of SEQ ID NO: 2; 2) the full length BSEP protein (SEQ ID NO:1), and any one or more of the peptide of SEQ ID NO: 2, the peptide of SEQ ID NO: 3, the peptide of SEQ ID NO: 4, the peptide of SEQ ID NO: 5, the peptide of SEQ ID NO: 6, the peptide of SEQ ID NO: 7, the peptide of SEQ ID NO: 8, or a combination thereof; 3) the peptide of SEQ ID NO: 2, and any one or more of the peptide of SEQ ID NO: 2, the peptide of SEQ ID NO: 3, the peptide of SEQ ID NO: 4, the peptide of SEQ ID NO: 5, the peptide of SEQ ID NO: 6, the peptide of SEQ ID NO: 7, the peptide of SEQ ID NO: 8, or a combination thereof; 4) any 2 or more of the peptide of SEQ ID NO: 2, the peptide of SEQ ID NO: 3, the peptide of SEQ ID NO: 4, the peptide of SEQ ID NO: 5, the peptide of SEQ ID NO: 6, the peptide of SEQ ID NO: 7, or the peptide of SEQ ID NO: 8, or a combination of thereof, or 5) the full length BSEP protein (SEQ ID NO:1), the peptide of SEQ ID NO: 2, and any one or more of the peptide of SEQ ID NO: 2, the peptide of SEQ ID NO: 3, the peptide of SEQ ID NO: 4, the peptide of SEQ ID NO: 5, the peptide of SEQ ID NO: 6, the peptide of SEQ ID NO: 7, the peptide of SEQ ID NO: 8, or a combination thereof.

According to some aspects, a method of determining or predicting the presence of BSEP deficiency in a subject includes detecting the presence and quantity of anti-BSEP antibodies in the biological sample of the subject, comparing the quantity of anti-BSEP antibodies from the biological sample to a quantity of anti-BSEP antibodies in a control sample or to a previously measured quantity of anti-BSEP antibodies from another biological sample of the subject, and determining whether the subject has BSEP deficiency or is likely to develop BSEP deficiency from the comparison.

In some aspects, a method of treating a subject having, or likely to have, a BSEP deficiency includes detecting the presence and quantity of anti-BSEP antibodies in the biological sample of the subject, determining whether the subject has BSEP deficiency or is likely to develop BSEP deficiency from the detecting, and if the subject is determined to have BSEP deficiency, administering a treatment selected from immunosuppressive therapy, plasmapheresis, immunoadsorption therapy, rituximab, or a combination thereof.

According to some aspects, a kit for detecting the presence and quantity of anti-BSEP antibodies includes a sample receiving region, and a surface comprising a test region comprising one or more immobilized BSEP peptides. In some embodiments, the sample receiving region, the surface, and test region are configured for enzyme-linked immunosorbent assay (ELISA).

### BRIEF DESCRIPTION OF THE DRAWINGS

These and other features and advantages of embodiments of the present disclosure will be better understood with reference to the following detailed description when considered in conjunction with the accompanying drawings, in which:

FIGS. 1A and 1B are schematics depicting the normal function of Bile Salt Export Pump (BSEP) in healthy human hepatocytes (FIG. 1A), and BSEP deficiency in patients having PFC2 (FIG. 1B);

FIG. 2A is a flowchart depicting a cascade of events leading to the development of Recurrent BSEP deficiency in a patient/subject with PFIC2;

FIG. 2B depicts a liver biopsy at the time of PFIC2 diagnosis;

FIG. 2C depicts a liver biopsy of a patient/subject having Recurrent BSEP deficiency taken post-liver transplant;

FIG. 3A is a Western blot of the serum of Patient 1 (diluted 1/1000) in the Examples, showing the presence of anti-BSEP autoantibodies;

FIG. 3B is a Western blot of the serum of Patient 2 (diluted 1/1000) in the Examples, showing the presence of anti-BSEP antibodies; and

FIG. 4 is a graph of the concentration of anti-BSEP autoantibodies against the full length BSEP peptide of the patients and the control in Example 8.

### DETAILED DESCRIPTION

### Definitions

Unless otherwise noted, terms are to be understood according to conventional usage by those of ordinary skill in the relevant art. In case of conflict, the present document, including definitions, will control. Exemplary methods and materials are described below, although methods and materials similar or equivalent to those described herein may be used in practice or testing of the present disclosure. All publications, patent applications, patents and other references mentioned herein are incorporated by reference in their entirety. The materials, methods, and examples disclosed herein are illustrative only and not intended to be limiting. The compositions and methods may comprise, consist of, or consist essentially of the elements of the compositions and/or methods as described herein, as well as any additional or optional element described herein or otherwise useful in the identification and/or treatment of patients having PFIC, PFIC2, or suffering from complications of a liver transplant.

As used herein and in the appended claims, the singular forms “a,” “an,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some aspects, the terms refer to humans. In further aspects, the terms may refer to adults or children, depending on the context.

Abbreviations for amino acids are used throughout this disclosure and follow the standard nomenclature known in the art. For example, as would be understood by those of ordinary skill in the art, Alanine is Ala or A; Arginine is Arg or R; Asparagine is Asn or N; Aspartic Acid is Asp or D; Cysteine is Cys or C; Glutamic acid is Glu or E; Glutamine is Gln or Q; Glycine is Gly or G; Histidine is His or H; Isoleucine is Ile or I; Leucine is Leu or L; Lysine is Lys or K; Methionine is Met or M; Phenylalanine is Phe or F; Proline is Pro or P; Serine is Ser or S; Threonine is Thr or T; Tryptophan is Trp or W; Tyrosine is Tyr or Y; and Valine is Val or V.

### Liver Transplant and Progressive Familial Intrahepatic Cholestasis (PFIC2)

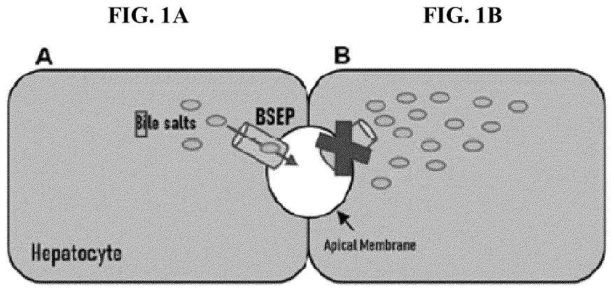

Progressive familial intrahepatic cholestasis 2 (PFIC2) is a hereditary disorder resulting from mutations in the ABCB11 (BSEP) gene, which is expressed in hepatocytes. As shown in FIG. 1, BSEP regulates bile acid flow by exporting bile acids into the bile canaliculi. BSEP deficiency can lead to cholestasis and liver failure. The standard treatment for severe liver disease in patients with PFIC2 is a liver transplant. But an estimated 8%-33% of transplant recipients develop alloimmunity post-transplant, i.e., generating antibodies against the donor liver graft. “Recurrent BSEP deficiency” and graft dysfunction are complications that can arise when transplant recipients develop these BSEP autoantibodies in the new liver graft. While the cause of these complications is generally established, their mechanism and a detection method for these BSEP autoantibodies remains unknown.

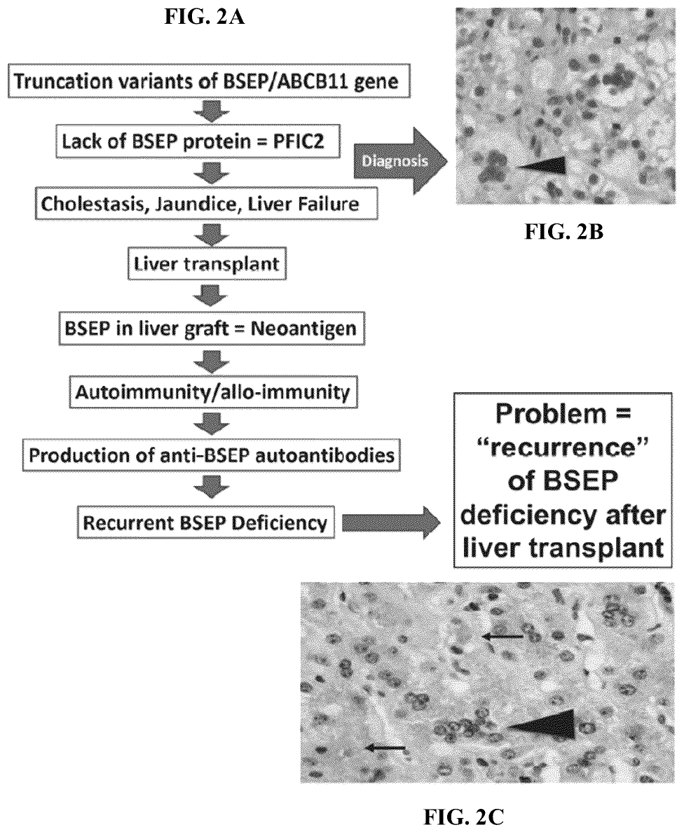

FIG. 2A is a flowchart depicting a cascade of events that can lead to Recurrent BSEP deficiency. As shown, mutations in the ABCB11 (BSEP) gene lead to a lack of BSEP protein, and a diagnosis of PFIC2. FIG. 2B shows a liver biopsy at the time of PFIC2 diagnosis, and shows multinucleated giant cell transformation (arrowheads). PFIC2 can lead to cholestasis, jaundice, or liver failure, which leads to liver transplant as the treatment. In PFIC2, the BSEP in the donor liver graft can act as a neoantigen, and the patient can develop autoimmunity/alloimmunity. This can lead to the production of anti-BSEP autoantibodies, which can cause Recurrent BSEP deficiency post-transplant. FIG. 2C shows a liver biopsy post-transplant, and histology shows cholestasis and the presence of multinucleated giant cell transformation (arrowheads).

According to embodiments of the present disclosure, a diagnostic method uses ELISA to detect and quantify autoantibodies against BSEP, a protein transporter that exports bile acids in the liver. The amount of anti-BSEP antibodies in a biological sample, such as a blood specimen or serum sample of a patient/subject, will determine the severity of autoimmune and alloimmune complications in such patients/subjects. This information can be used to support clinicians in making medical decisions, making diagnoses, and choosing/adjusting medications and/or treatment protocols.

By using various epitopes of BSEP, embodiments of the present disclosure can characterize the anti-BSEP antibodies in the patient/subject, and can detect and quantify sub-type antibodies reacting to specific parts of the full length BSEP protein. This can allow physicians to identify the target epitope of BSEP, and monitor the patient's/subject's levels of autoantibodies to better prepare for any developing setbacks, including rejection or dysfunction of the donor liver organ, or neutralization, destruction, or interference of transgenes, and to tackle oncoming complications before they arise, e.g., by increasing immunosuppression, or changing the types or protocols (including dose and duration) of immunosuppression medications. Also, in severe cases, antibody depletion/removal therapies—such as plasmapheresis or immunoadsorption—may be indicated. Immunosuppression has improved graft survival, but may leave the patient susceptible to infectious complications. Allograft rejection is mediated primarily by T cells, with B cells playing a role via antibody production. Depending on the transplant type, rejection can be hyperacute, acute, or chronic. Any of these can be life-threatening. Therefore, early detection of antibodies is critical to change the clinical course by providing effective early treatment.

As noted above, recipients of gene therapy or messenger RNA therapy may induce autoimmunity to the “corrected” BSEP after treatment. In such cases of gene therapy for PFIC2 patients, the methods according to embodiments of the present disclosure can detect developing autoantibodies against the product of the induced transgene. These methods, therefore, can be a powerful tool to diagnose this serious complication.

According to aspects of the present disclosure, a reliable diagnostic method detects the presence of anti-BSEP autoantibodies, and quantifies the autoantibodies in a biological sample. The methods according to embodiments of the present disclosure can be used to detect the presence of, and monitor concentrations of the autoantibodies, to thereby indicate, determine, or predict the presence of a BSEP deficiency and/or to indicate, determine, or predict whether the subject (from which the biological sample is taken) has or is likely to develop recurrent BSEP deficiency. In some aspects, the methods may also be used to inform treatment protocols for minimizing recurrent BSEP deficiency.

Indeed, according to aspects, the methods disclosed herein allow for improved treatment modalities aimed at minimizing recurrent BSEP deficiency. For example, once it has been quantified just how much the patient's antibodies are “attacking” the transplant or transgenes, the treating physician or medical team can alter the treatment plan to quickly improve the patient's condition, such as increasing the amount of immunosuppression, or altering the type and duration of treatment. An optimal immunosuppressive regimen will maintain liver function, and minimize rejection, while limiting the potential for infection. Thus, aspects of the present disclosure provide the development of individual treatment plans for these patients/subjects, which can adapt to their particular condition in a much more efficient, facile, and safe manner than has previously been done.

To that end, any suitable, known or future treatment for BSEP deficiency may be used. For example, in some embodiments, if the method determines or predicts the presence of a BSEP deficiency or a likelihood of developing such a BSEP deficiency or recurrent BSEP deficiency post-transplant, the treatment protocol may involve administering immunosuppressive therapy, changing existing immunosuppressive therapy protocols, administering plasmapheresis or immunoadsorption therapies to deplete the anti-BSEP antibodies, or administering certain monoclonal antibodies, such as rituximab—a monoclonal antibody against CD20. Such treatment protocols may enable reductions in, or eliminations of the BSEP deficiency, which may lead to prevention of, or a reduction in the likelihood of developing graft dysfunction due to BSEP deficiency post-transplant. And to the extent the patient/subject may have already experienced some graft dysfunction, the methods according to aspects of the present disclosure may be used to diagnose the cause of the graft dysfunction, and therefore inform treatment protocols for reducing or eliminating the BSEP deficiency (as discussed herein) to thereby remedy, halt, or reduce the dysfunction, and save the donor liver.

In some aspects, the methods can be used to monitor the progress of liver transplant patients post-transplant. For example, the methods may be used to indicate, determine, or predict whether the patient/subject is likely to, or has begun to, develop BSEP deficiency or recurrent BSEP deficiency, which can lead to dysfunction of the donor liver graft. The methods can also be used pre-transplant to indicate, determine, or predict whether the patient/subject is likely to develop BSEP deficiency in the new donor liver post-transplant. Such a pre-transplant assay can inform on treatment protocols, which when delivered early (e.g., pre-transplant, and/or immediately post-transplant) can prevent, or reduce the likelihood of the patient/subject developing BSEP deficiency in the donor liver graft post-transplant.

In some aspects of the present disclosure, a method of detecting the presence and quantity of anti-BSEP antibodies in a biological sample from a subject/patient includes contacting the biological sample with one or more BSEP peptides to form an antibody-BSEP peptide complex, and detecting the amount of the antibody-BSEP peptide complex in the biological sample. In some aspects, the BSEP peptide is immobilized on a solid support, and the contacting includes introducing the biological sample to the BSEP peptide on the solid support. In some aspects, the detecting the presence and quantity of the anti-BSEP antibodies are carried out via enzyme-linked immunosorbent assay (ELISA).

In some embodiments, the one or more BSEP peptides may include one or more of the full length BSEP protein (SEQ ID NO:1; including amino acids (aa) 1-1321), and/or one or more epitopes of BSEP. In some aspects, the one or more epitopes of BSEP may be selected from: a “Sigma” peptide (SEQ ID NO: 2; aa 616-aa 746 of the full length BSEP protein sequence; available as APREST86066 from Sigma-Aldrich); and/or one or more peptide segments of the full length BSEP protein sequence having a length of 18-20 amino acids; and/or combinations thereof. For example, in some aspects, the one or more epitopes may be selected from the Sigma BSEP peptide (SEQ ID NO: 2; including aa 616-aa 746 of the full length BSEP protein sequence), Peptide 1 (SEQ ID NO: 3; including aa 3-aa 22 of the full length BSEP protein sequence), Peptide 2 (SEQ ID NO: 4; including aa 165-aa 184 of the full length BSEP protein sequence), Peptide 3 (SEQ ID NO: 5; including aa 534-aa 553 of the full length BSEP protein sequence), Peptide 4 (SEQ ID NO: 6; including aa 1194-aa 1213 of the full length BSEP protein sequence), Peptide 5 (SEQ ID NO: 7; including aa 1214-aa 1233 of the full length BSEP protein sequence), Peptide 6 (SEQ ID NO: 8; including aa 1302-aa 1321 of the full length BSEP protein sequence), and/or any combinations thereof.

The one or more BSEP peptide may include a single peptide or a panel of 2 or more peptides. For example, in some embodiments, the single BSEP peptides may include one of the full length BSEP peptide (SEQ ID NO: 1), the Sigma peptide (SEQ ID NO: 2), or any one of Peptide 1 (SEQ ID NO: 3), Peptide 2 (SEQ ID NO: 4), Peptide 3 (SEQ ID NO: 5), Peptide 4 (SEQ ID NO: 6), Peptide 5 (SEQ ID NO: 7), and/or Peptide 6 (SEQ ID NO: 8).

In some embodiments, the panel of 2 or more BSEP peptides may include the full length BSEP peptide (SEQ ID NO: 1), and the Sigma peptide (SEQ ID NO: 2). And in some embodiments, the panel may include the full length BSEP protein (SEQ ID NO:1), and any one or more of Peptide 1 (SEQ ID NO: 3), Peptide 2 (SEQ ID NO: 4), Peptide 3 (SEQ ID NO: 5), Peptide 4 (SEQ ID NO: 6), Peptide 5 (SEQ ID NO: 7), and/or Peptide 6 (SEQ ID NO: 8). In some embodiments, the panel may include the Sigma peptide (SEQ ID NO: 2), and any one or more of Peptide 1 (SEQ ID NO: 3), Peptide 2 (SEQ ID NO: 4), Peptide 3 (SEQ ID NO: 5), Peptide 4 (SEQ ID NO: 6), Peptide 5 (SEQ ID NO: 7), and/or Peptide 6 (SEQ ID NO: 8). In some embodiments, the panel may include any 2 or more of Peptide 1 (SEQ ID NO: 3), Peptide 2 (SEQ ID NO: 4), Peptide 3 (SEQ ID NO: 5), Peptide 4 (SEQ ID NO: 6), Peptide 5 (SEQ ID NO: 7), and/or Peptide 6 (SEQ ID NO: 8). And in some embodiments, the panel may include the full length BSEP protein (SEQ ID NO:1), the Sigma peptide (SEQ ID NO: 2), and any one or more of Peptide 1 (SEQ ID NO: 3), Peptide 2 (SEQ ID NO: 4), Peptide 3 (SEQ ID NO: 5), Peptide 4 (SEQ ID NO: 6), Peptide 5 (SEQ ID NO: 7), and/or Peptide 6 (SEQ ID NO: 8).

The amino acid sequence for the full BSEP peptide is the following:

(SEQ ID NO: 1)
MSDSVILRSIKKFGEENDGFESDKSYNNDKKSRLQDEKKGDGVRV
GFFQLFRFSSSTDIWLMFVGSLCAFLHGIAQPGVLLIFGTMTDVF
IDYDVELQELQIPGKACVNNTIVWTNSSLNQNMTNGTRCGLLNIE
SEMIKFASYYAGIAVAVLITGYIQICFWVIAAARQIQKMRKFYFR
RIMRMEIGWFDCNSVGELNTRFSDDINKINDAIADQMALFIQRMT
STICGFLLGFFRGWKLTLVIISVSPLIGIGAATIGLSVSKFTDYE
LKAYAKAGVVADEVISSMRTVAAFGGEKREVERYEKNLVFAQRWG
IRKGIVMGFFTGFVWCLIFLCYALAFWYGSTL VLDEGEYTPGTL
VQIFLSVIVGALNLGNASPCLEAFATGRAAATSIFETIDRKPIID
CMSEDGYKLDRIK GEIEFHNVTFHYPSRPEVKILNDLNMVIKPG
EMTALVGPSGAGKSTALQLIQRFYDPCEGMVTVDGHDIRSLNIQW
LRDQIGIVEQEPVLFSTTIAENIRYGREDATMEDIVQAAKEANAY
NFIMDLPQQFDTLVGEGGGQMSGGQKQRVAIARALIRNPKILLLD
MATSALDNESEAMVQEVLSKIQHGHTIISVAHRLSTVRAADTIIG
FEHGTAVERGTHEELLERKGVYFTLVTLQSQGNQALNEEDIKDAT
EDDMLARTFSRGSYQDSLRASIRQRSKSQLSYLVHEPPLAVVDHK
STYEEDRKDKDIPVQEEVEPAPVRRILKFSAPEWPYMLVGSVGAA
VNGTVTPLYAFLFSQILGTFSIPDKEEQRSQINGVCLLFVAMGCV
SLFTQFLQGYAFAKSGELLTKRLRKFGFRAMLGQDIAWFDDLRNS
PGALTTRLATDASQVQGAAGSQIGMIVNSFTNVTVAMIIAFSFSW
KLSLVILCFFPFLALSGATQTRMLTGFASRDKQALEMVGQITNEA
LSNIRTVAGIGKERRFIEALETELEKPFKTAIQKANIYGFCFAFA
QCIMFIANSASYRYGGYLISNEGLHFSYVFRVISAVVLSATALGR
AFSYTPSYAKAKISAARFFQLLDRQPPISVYNTAGEKWDNFQGKI
DFVDCKFTYPSRPDSQVLNGLSVSISPGQTLAFVGSSGCGKSTSI
QLLERFYDPDQGKVMIDGHDSKKVNVQFLRSNIGIVSQEPVLFAC
SIMDNIKYGDNTKEIPMERVIAAAKQAQLHDFVMSLPEKYETNVG
SQGSQLSRGEKQRIAIARAIVRDPKILLLDEATSALDTESEKTVQ
VALDKAREGRTCIVIAHRLSTIQNADIIAVMAQGVVIEKGTHEEL
MAQKGAYYKLVTTGSPIS

A link for the full length BSEP protein sequence can be found at useast.ensembl.org/Homo_sapiens/Gene/Summary?db=core;g=ENSG00000073734;r=2:168915498-169031324;t=ENST00000650372, or here: www.ncbi.nlm.nih.gov/nuccore/NM_003742.4.

The following Table 1 lists the amino acid sequence for the Sigma Peptide, and Peptides 1 through 6, all of which are epitopes of the full length BSEP protein.

TABLE 1

| | | LOCATION IN | LENGTH | |
|---|---|---|---|---|
| | | FULL LENGTH | OF | |
| PEPTIDE | SEQ ID NO | BSEP PROTEIN | SEQUENCE | SEQUENCE |
| Sigma | SEQ ID NO: 2 | aa 616-aa 746 | 131 | RLSTVRAADT |
| Peptide | | | amino | IIGFEHGTAV |
| | | | acids | ERGTHEELLE |
| | | | | RKGVYFTLVT |
| | | | | LQSQGNQALN |
| | | | | EEDIKDATED |
| | | | | DMLARTFSRG |
| | | | | SYQDSLRASI |
| | | | | RQRSKSQLSY |
| | | | | LVHEPPLAVV |
| | | | | DHKSTYEEDR |
| | | | | KDKDIPVQEE |
| | | | | VEPAPVRRIL |
| | | | | K |
| Peptide 1 | SEQ ID NO: 3 | aa 3-aa 22 | 20 | DSVILRSIKK |
| | | | amino | FGEENDGFES |
| | | | acids | |
| Peptide 2 | SEQ ID NO: 4 | aa 165-aa 184 | 20 | IAAARQIQKM |
| | | | amino | RKFYFRRIMR |
| | | | acids | |
| Peptide 3 | SEQ ID NO: 5 | aa 534-aa 553 | 20 | EANAYNFIMD |
| | | | amino | LPQQFDTLVG |
| | | | acids | |
| Peptide 4 | SEQ ID NO: 6 | aa 1194-aa 1213 | 20 | QAQLHDFVMS |
| | | | amino | LPEKYETNVG |
| | | | acids | |
| Peptide 5 | SEQ ID NO: 7 | aa 1214-aa 1233 | 20 | SQGSQLSRGE |
| | | | amino | KQRIAIARAI |
| | | | acids | |
| Peptide 6 | SEQ ID NO: 8 | aa 1302-aa 1321 | 20 | ELMAQKGAYY |
| | | | amino | KLVTTGSPIS |
| | | | acids | |

The biological sample used in the methods disclosed herein is not particularly limited, and may be any suitable biological sample from the target patient/subject. In some aspects, the biological sample may be selected from blood, urine, saliva, cerebrospinal fluid, tissue biopsies, swabs (nasal, throat, or buccal), plasma, serum, and combinations thereof. In some aspects, the biological sample is blood, plasma, serum, or a combination thereof. For example, in some aspects, the biological sample is serum. In aspects, the biological sample is freshly obtained (e.g., never frozen, and/or obtained within an hour of testing) at the time of subjecting the sample to the disclosed methods.

Methods of preparing biological samples for detection assays are known, and the sample obtained from the patient/subject can be subjected to one or more processes prior to detection. Non-limiting exemplary methods include: centrifugation to separate plasma or serum from blood; filtration or centrifugation to remove particulates from urine; collection and possible dilution or stabilization of saliva samples using specialized devices; filtration of cerebrospinal fluid collected via lumbar puncture; homogenization or lysis of tissue biopsies to release cellular contents; elution of swabs in buffer solutions; and treatments such as dilution, filtration, or enzymatic digestion for other bodily fluids. In some aspects, the sample is a blood sample, and is not subjected to any processes prior to detection of the anti-BSEP antibodies. And in some aspects, the sample is a serum sample, which may be obtained from a whole blood sample collected from the patient/subject which is then subjected to a separation process to separate the serum from the whole blood.

According to some aspects of the present disclosure, an in-vitro method of determining or predicting the presence of BSEP deficiency in a subject/patient includes detecting and quantifying a level or amount of anti-BSEP antibodies in a biological sample of the subject/patient, and comparing the level or amount of anti-BSEP antibodies from the biological sample to the level or amount of anti-BSEP antibodies in a normal or control sample or to a previously measured level or amount of anti-BSEP antibodies from another biological sample of the subject/patient. When the comparison reveals that the detected level or amount of anti-BSEP antibodies is near or about the level or amount in the normal or control sample or to the previously measured level or amount, the method indicates that the subject/patient does not have BSEP deficiency, or that the patient/subject is recovering from a prior BSEP deficiency. And in some embodiments, when the comparison reveals that the detected level or amount of anti-BSEP antibodies is higher than the level or amount in the normal or control sample or to the previously measured level or amount, the method indicates that the subject/patient has, is developing, or is likely to develop BSEP deficiency.

The detecting and quantifying the anti-BSEP antibodies are carried out according to the methods described herein. For example, the detecting and quantifying includes contacting a biological sample from the patient/subject with the one or more BSEP peptides described herein to form an antibody-BSEP peptide complex, and detecting the amount of the antibody-BSEP peptide complex in the biological sample. As also disclosed elsewhere herein, in some aspects, the detecting and quantifying are carried out via ELISA.

In some embodiments, the detecting and comparing levels or amounts of anti-BSEP antibodies in the subject/patient may be conducted multiple times over a specified period of time in order to monitor the BSEP function of the subject/patient. Such repetitive monitoring enables the physician to determine whether the measured levels of anti-BSEP antibodies are remaining the same, reducing, or increasing over time post-transplant. By comparing the results of these repetitive measurements over time, the method can inform treatment protocols for reducing recurrent BSEP deficiency which can lead to donor graft dysfunction or further transplant complications. For example, the level or amount of anti-BSEP antibodies remaining the same or increasing over time may be indicative of the patient/subject having either recurrent BSEP deficiency or other transplant complications, enabling the physician to prescribe appropriate corrective or treatment protocols. Conversely, the level or amount of anti-BSEP antibodies reducing over time may be indicative of the patient/subject returning to normal BSEP function.

While embodiments of the methods disclosed herein are described generally as detecting or monitoring BSEP levels or deficiency pre- or post-liver transplant in a subject, it is understood that these methods may be used in any suitable context. For example, the methods may be used, as discussed above, for the detection of BSEP antibodies in connection with a planned or completed liver transplant in an effort to inform potential treatment protocols and transplant management in patients/subjects identified as having or likely to develop BSEP deficiency. However, the methods may also be used to detect and quantify anti-BSEP antibodies in any patients/subjects, regardless of any underlying condition or disease, and also may be used in the context of other therapies, e.g., in patients/subject that have received or are planned to receive gene therapy, protein expression/replacement therapy, and messenger RNA therapy.

In further aspects of the present disclosure, a method is provided for treating a patient/subject having, or likely to have, a BSEP deficiency, as determined by the detection and quantification of anti-BSEP antibodies in a sample obtained from the patient/subject. Based on this detection and quantification of anti-BSEP antibodies, a treatment for BSEP deficiency is administered to the patient/subject. In some aspects, the patient/subject having or likely to have a BSEP deficiency may be a patient/subject having, or likely to have PFIC2. And in some aspects, the patient/subject having or likely to have a BSEP deficiency maybe a patient/subject that is planned to have, or has already undergone, a liver transplant.

According to some aspects, a method is provided for treating graft dysfunction in a post-liver transplant patient/subject having, or likely to have, a BSEP deficiency, as determined by the detection and quantification of anti-BSEP antibodies in a sample obtained from the patient/subject. Based on this detection and quantification of anti-BSEP antibodies, a treatment for BSEP deficiency is administered to the patient/subject.

In further aspects, kits for the detection of anti-BSEP antibodies are disclosed, the kits being useful for the diagnosis and/or treatment of BSEP deficiency in a patient/subject in need thereof. The patient/subject is not particularly limited, but in some embodiments, may be a pediatric patient. In some aspects, the patient/subject may be an adult patient. In some aspects, the patient/subject may be one presenting with one or more symptoms of PFIC2, BSEP deficiency, or liver graft dysfunction post-transplant. In aspects, the patient/subject may be suspected of having a BSEP deficiency, a recurrent BSEP deficiency, or liver graft dysfunction post-transplant. In some aspects, the patient/subject may be one diagnosed with or likely to have BSEP deficiency, or PFIC2, and planned for, or has already undergone a liver transplant.

In some aspects, the kit may be designed for ELISA. ELISA methods are generally known in the art. In brief, an antibody is used to bind the biomarker of interest. A biological sample, such as blood, urine, cell lysate, or serum, is introduced to a surface coated with a capture antibody specific to the target biomarker. After binding, a secondary antibody conjugated to an enzyme (such as, for example, horseradish peroxidase (HRP)) is added. Upon substrate introduction, the enzyme catalyzes a reaction that produces a detectable signal, typically colorimetric or chemiluminescent, which correlates with the biomarker's concentration in the sample. ELISA can be used for either or both quantitative and/or qualitative detection. According to aspects of the present disclosure, the ELISA kit includes a surface coated with the one or more BSEP peptides described herein, and the target biomarkers are the anti-BSEP antibodies.

In some aspects, the surface coated with the one or more BSEP peptides includes a sample receiving region and a capture surface comprising the one or more BSEP peptides immobilized on the capture surface. The sample receiving region is designed to accommodate and direct the biological sample towards the capture surface. The sample receiving region may also be configured to handle various types of biological samples, such as blood, urine, saliva, tissue extracts, and combinations thereof. This region may include features such as wells, channels, or pads to ensure efficient application and distribution of the sample.

Upon application of the biological sample to the sample receiving region, the biological sample is directed to the capture surface where the one or more anti-BSEP antibodies, if present, bind to the immobilized one or more BSEP peptides. This binding event generates a detectable signal, which can be measured, e.g., using a colorimetric assay to produce a visible color change, or a fluorescence (or chemiluminescent) assay to produce a fluorescence (or light-based) signal.

### EXAMPLES

The following non-limiting examples are provided to further illustrate embodiments of the present disclosure. It should be appreciated by those of skill in the art that the techniques disclosed in the examples that follow represent approaches that have been found to function well in the practice of the disclosure, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the disclosure.

### Materials and Methods

ELISA: The serum or plasma of two PFIC2 patients and healthy controls were used in ELISA assays to detect and quantify autoantibody concentration against seven different epitopes of BSEP (SEQ ID NOs: 2-8) or against full-length BSEP (SEQ ID NO: 1).

Epitopes: The seven epitopes of BSEP were: 1) APREST86066 obtained from Sigma-Aldrich, which is a 131 amino acid long peptide corresponding to aa 616 through aa 746 of the full-length BSEP peptide (“Sigma” peptide; SEQ ID NO: 2); 2) Peptide 1, a 20 amino acid long epitope corresponding to aa 3 through aa 22 the full-length BSEP peptide (SEQ ID NO: 3); 3) Peptide 2, a 20 amino acid long epitope corresponding to aa 165 through aa 184 of the full-length BSEP peptide (SEQ ID NO:4); 4) Peptide 3, a 20 amino acid long epitope corresponding to aa 534 through aa 553 of the full-length BSEP peptide (SEQ ID NO: 5); 5) Peptide 4, a 20 amino acid long epitope corresponding to aa 1194 through aa 1213 of the full-length BSEP peptide (SEQ ID NO: 6); 6) Peptide 5, a 20 amino acid long epitope corresponding to aa 1214 through aa 1233 of the full-length BSEP peptide (SEQ ID NO: 7); and 7) Peptide 6, a 20 amino acid long epitope corresponding to aa 1302 through 1321 of the full-length BSEP peptide (SEQ ID NO: 8). Peptides 1 through 6 (SEQ ID NOs: 3-8) are custom peptides identified using a datasheet on epitopes supplied by Immune Epitope Database and Analysis Resource (IEDB), and were synthesized with GenScript. The full-length BSEP peptide was made in HEK cells by plasmid transfection (APREST86066 obtained from Sigma-Aldrich). An anti-ABCB11 antibody produced in rabbits against BSEP (HPA019035 obtained from Sigma-Aldrich) was used to generate the standard reaction reference.

Chemical Materials: 1) Full-length BSEP peptide—commercially available Human BSEP peptide (recombinant) from Sigma-Aldrich; 2) Carbonate-Bicarbonate coating buffer capsule—commercially available as C3041 from Sigma-Aldrich, 1 capsule/100 ml deionized water; 3) Tris Buffered Saline with Tween 20—commercially available as T9039 from Sigma-Aldrich, 1 packet/1 L deionized water; 4) Rabbit Anti-ABCB11 antibody—HPA019035 Prestige Antibodies commercially available from Sigma-Aldrich, 01.mg/ml; 5) 50 mM Tris Buffering Saline—commercially available as T6789 from Sigma Chemical, pH 8.0, 1% BSA, 1 packet/1 L deionized water; 6) 10% Tween 20—commercially available as E108 from Bethyl Laboratories; 7) Mouse Monoclonal Anti-Rabbit IgG Biotin—commercially available as sc-2491 from Santa Cruz Biotechnology, 400 ug/ml; 8) Recombinant Rabbit Monoclonal Anti-Human IgG Biotin—commercially available as ab201485 from Abcam Ltd., 500 ug/ml; 9) Streptavidin-HRP commercially available as ab7403 from Abcam Ltd. 1/3000 dilution; 10) TMB (HRP Microwell Substrate)—commercially available as E102 from Bethyl Laboratories; 11) ELISA Stop Solution—commercially available as E115 from Bethyl Laboratories.

Biological characteristics: Antibody protein interactions detected by Horseradish peroxidase with 3,3′,5,5′-Tetramethylbenzidine (TMB) substrate reaction, showing colour change.

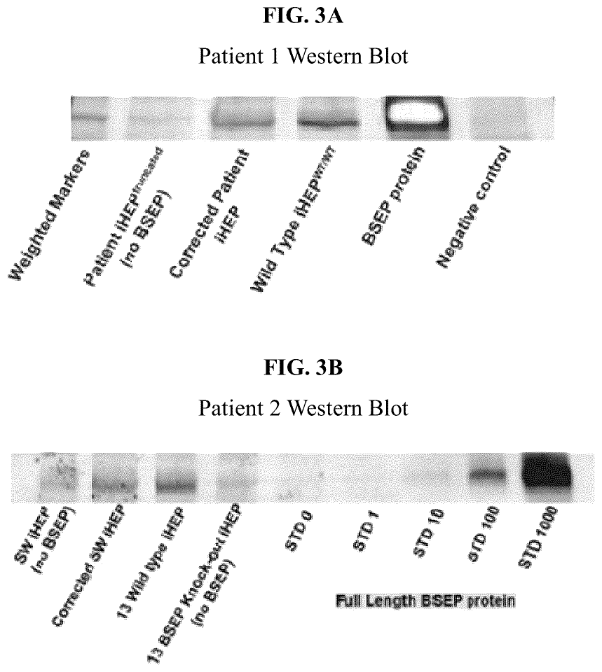

Procedure: The portion of the BSEP amino acid sequence was coated on a 96-well plate. Serum of two patients having recurrent BSEP deficiency were analysed. As shown in FIGS. 3A and 3B, both patients had confirmed anti-BSEP autoantibodies in their serum from previously conducted Western blotting (FIG. 3A depicts patient 1, and FIG. 3B depicts patient 2). Using serum from Patient 1 and Patient 2, the concentration of autoantibodies to the selection portion of the BSEP peptide was detected and quantified.

### Example 1

The ELISA was run using the Sigma peptide (SEQ ID NO: 2) coated on the well plate. Patient 1 was tested three times: 1) pre-plasmapheresis treatment; 2) post-plasmapheresis treatment; and 3) according to an annual follow-up blood test. Table 2 below shows the results of the quantification for each of Patient 1's measurements, as well as the measurements for Patent 2 and the 4 controls.

TABLE 2
Example 1 Antibody Quantification Results -
Sigma Peptide (SEQ ID NO: 2)

| | Concentration |
|---|---|
| Serum Sample | of Antibodies (μg/mL) |
| Patient 1, pre-plasmapheresis | 0.840 |
| Patient 1, post-plasmapheresis | 0.000 |
| Patient 1, annual follow-up | 0.010 |
| Patient 2 | 0.000 |
| Control 1 | 0.000 |
| Control 2 | 0.004 |
| Control 3 | 0.726 |
| Control 4 | 4.507 |

The significant presence of certain autoantibodies was found in the control serum, suggesting that those specific autoantibodies do not contribute to clinical manifestations. As seen here, Patient 2 does not show anti-BSEP autoantibodies against the Sigma epitope of BSEP, showing that BSEP deficiency can still occur in the absence of autoantibodies against this epitope of BSEP.

### Examples 2 Through 7

The ELISA was run using the custom peptides (Peptides 1 through 6; SEQ ID NOs: 3-8) coated on the well plate, respectively. Table 3 below shows the results of the quantification for Patient 1 and Patient 2.

TABLE 2
Examples 2-7 Antibody Quantification Results -
Peptides 1-6 (SEQ ID NOs: 3-8)

| | | Patient 1 | Patient 2 |
|---|---|---|---|
| | | Concentration | Concentration |
| | | of Antibodies | of Antibodies |
| Example | Peptide | (μg/mL) | (μg/mL) |
| 2 | 1 (SEQ ID NO: 3) | 71.34 | 7.82 |
| 3 | 2 (SEQ ID NO: 4) | 35.24 | 4.17 |
| 4 | 3 (SEQ ID NO: 5) | 388.86 | 6.42 |
| 5 | 4 (SEQ ID NO: 6) | 185.68 | 18.37 |
| 6 | 5 (SEQ ID NO: 7) | 53.35 | 7.60 |
| 7 | 6 (SEQ ID NO: 8) | 6.42 | 10.33 |

As can be seen in Table 2, Patient 1 shows significantly higher concentrations of autoantibodies than Patient 2.

### Example 8

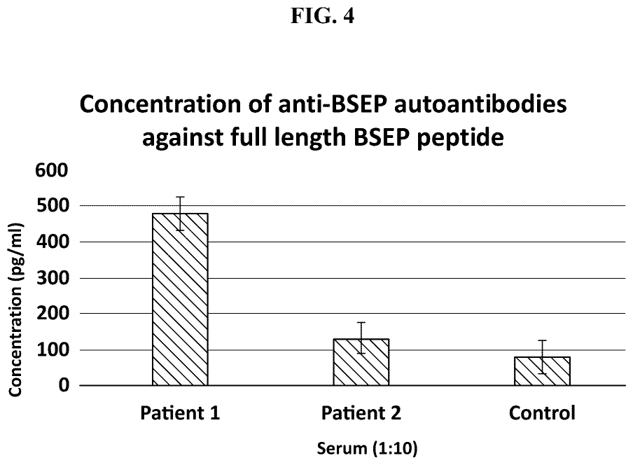

The ELISA was run using the full-length BSEP peptide (SEQ ID NO: 1) coated on the well plate. FIG. 4 is a graph depicting the results of the quantification for Patient 1 and Patient 2 measured against control. As shown in FIG. 4, Patient 1 shows significantly higher concentrations of autoantibodies, while Patient 2 shows higher concentrations than control but not significant concentrations of autoantibodies.

As shown in the above Examples, the ELISA of Patients 1 and 2, and healthy sera successfully quantified the concentration of anti-BSEP autoantibodies. This demonstrates the efficacy of the methods according to aspects of the present disclosure.

Additionally, the method of detecting concentrations of anti-BSEP autoantibodies in a biological sample may be affected by the specific anti-BSEP autoantibody, and whether it is capable of specifically binding to at least one epitope of BSEP. Accordingly, in some embodiments, as discussed above, the methods may include using a panel of peptides to detect and quantify the autoantibodies. As also discussed above, this panel of peptides may include any one or more, or two or more of SEQ ID NO: 1—the full length BSEP peptide (aa 1-aa 1321), SEQ ID NO: 2—a Sigma BSEP peptide (aa 616-aa 746), SEQ ID NO: 3—Peptide 1 (aa 3-aa 22), SEQ ID NO: 4—Peptide 2 (aa 3-aa 22), SEQ ID NO: 5—Peptide 3 (aa 534-aa 553), SEQ ID NO: 6—Peptide 4 (aa 1194-aa 1213), SEQ ID NO: 7—Peptide 5 (aa 1214-aa 1233), and/or SEQ ID NO: 6—Peptide 6 (aa 1302-aa 1321).

The detection and testing methods disclosed herein can detect and quantify anti-BSEP antibodies in a biological sample, such as a patient's serum, blood, or plasma. These methods can be used to quantify and characterize ant-BSEP antibodies after liver transplant, messenger RNA therapy, or gene therapy, and can aid in diagnosing and treating patients/subjects with post-transplant or post-transgene therapy (gene therapy or messenger RNA therapy) liver complications, monitoring the condition of the patient/subject when the patient/subject is recovering from liver transplantation or rejection, and monitoring the efficacy of treatment.

While certain exemplary embodiments of the present disclosure have been illustrated and described, those of ordinary skill in the art will recognize that various changes and modifications can be made to the described embodiments without departing from the spirit and scope of the present disclosure, and equivalents thereof, as defined in the claims that follow this description. For example, although certain components may have been described in the singular, i.e., “a” BSEP peptide, and the like, one or more of these components in any combination can be used according to the present disclosure.

Although certain embodiments have been described as “comprising” or “including” the specified components, embodiments “consisting essentially of” or “consisting of” the listed components are also within the scope of this disclosure. For example, while embodiments of the present disclosure are described as including one or more of certain BSEP peptides, methods consisting essentially of or consisting of the noted BSEP peptides are also within the scope of this disclosure. Accordingly, the method may consist essentially of the noted BSEP peptides. In this context, “consisting essentially of” means that any additional components used in the method for detection and quantification will not materially affect the ability of methods to accurately detect and quantify anti-BSEP antibodies in the biological sample.

As used herein, unless otherwise expressly specified, all percentages and ratios are calculated by weight unless otherwise indicated. And all percentages and ratios are calculated based on the total composition unless otherwise indicated.

All numbers such as those expressing values, ranges, amounts or percentages may be read as if prefaced by the word “about,” even if the term does not expressly appear. Further, the word “about” is used as a term of approximation, and not as a term of degree, and reflects the penumbra of variation associated with measurement, significant figures, and interchangeability, all as understood by a person having ordinary skill in the art to which this disclosure pertains. Any numerical range recited herein is intended to include all sub-ranges subsumed therein. Plural encompasses singular and vice versa. For example, where the present disclosure describes “a” BSEP peptide, a mixture of such peptides can be used. When ranges are given, any endpoints of those ranges and/or numbers within those ranges can be combined within the scope of the present disclosure. The terms “including” and like terms mean “including but not limited to,” unless specified to the contrary.

Notwithstanding that the numerical ranges and parameters set forth herein may be approximations, any numerical value inherently contains certain errors necessarily resulting from the standard variation found in their respective testing measurements. The word “comprising” and variations thereof as used in this description and in the claims do not limit the disclosure to exclude any variants or additions.

Every document cited herein, including any cross-referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

1. A method of detecting the presence and quantity of anti-BSEP antibodies in a biological sample from a subject, the method comprising:
contacting the biological sample with one or more BSEP peptides to form an antibody-BSEP peptide complex, and
detecting the amount of the antibody-BSEP peptide complex in the biological sample.

2. The method according to claim 1, wherein the BSEP peptide is immobilized on a solid support, and the contacting includes introducing the biological sample to the BSEP peptide on the solid support.

3. The method according to claim 1, wherein the detecting the presence and quantity of the anti-BSEP antibodies are carried out via enzyme-linked immunosorbent assay (ELISA).

4. The method according to claim 1, wherein the one or more BSEP peptides comprises one or more of a full length BSEP peptide (SEQ ID NO:1), or one or more epitopes of BSEP.

5. The method of claim 4, wherein the one or more epitopes of BSEP are selected from the group consisting of the peptide of SEQ ID NO: 2, one or more epitopes of the full length BSEP peptide having a length of 18-20 amino acids, and combinations thereof.

6. The method of claim 4, wherein the one or more epitopes are selected from the group consisting of the peptide of SEQ ID NO: 2, the peptide of SEQ ID NO: 3, the peptide of SEQ ID NO: 4, the peptide of SEQ ID NO: 5, the peptide of SEQ ID NO: 6, the peptide of SEQ ID NO: 7, the peptide of SEQ ID NO: 8, and combinations thereof.

7. The method of claim 1, wherein the one or more BSEP peptides comprises a single BSEP peptide.

8. The method of claim 1, wherein the single BSEP peptide is selected from the group consisting of the full length BSEP peptide (SEQ ID NO: 1), the peptide of SEQ ID NO: 2, the peptide of SEQ ID NO: 3, the peptide of SEQ ID NO: 4, the peptide of SEQ ID NO: 5, the peptide of SEQ ID NO: 6, the peptide of SEQ ID NO: 7, or the peptide of SEQ ID NO: 8.

9. The method of claim 1, wherein the one or more BSEP peptides comprises a panel of 2 or more peptides.

10. The method of claim 9, wherein the panel of 2 or more BSEP peptides comprises one of:
the full length BSEP peptide (SEQ ID NO: 1), and the peptide of SEQ ID NO: 2;
the full length BSEP protein (SEQ ID NO:1), and any one or more of the peptide of SEQ ID NO: 2, the peptide of SEQ ID NO: 3, the peptide of SEQ ID NO: 4, the peptide of SEQ ID NO: 5, the peptide of SEQ ID NO: 6, the peptide of SEQ ID NO: 7, the peptide of SEQ ID NO: 8, or a combination thereof;
the peptide of SEQ ID NO: 2), and any one or more of the peptide of SEQ ID NO: 2, the peptide of SEQ ID NO: 3, the peptide of SEQ ID NO: 4, the peptide of SEQ ID NO: 5, the peptide of SEQ ID NO: 6, the peptide of SEQ ID NO: 7, the peptide of SEQ ID NO: 8, or a combination thereof;
any 2 or more of the peptide of SEQ ID NO: 2, the peptide of SEQ ID NO: 3, the peptide of SEQ ID NO: 4, the peptide of SEQ ID NO: 5, the peptide of SEQ ID NO: 6, the peptide of SEQ ID NO: 7, or the peptide of SEQ ID NO: 8, or a combination of thereof, or the full length BSEP protein (SEQ ID NO:1), the peptide of SEQ ID NO: 2, and any one or more of the peptide of SEQ ID NO: 2, the peptide of SEQ ID NO: 3, the peptide of SEQ ID NO: 4, the peptide of SEQ ID NO: 5, the peptide of SEQ ID NO: 6, the peptide of SEQ ID NO: 7, the peptide of SEQ ID NO: 8, or a combination thereof.

11. A method of determining or predicting the presence of BSEP deficiency in a subject, the method comprising:
detecting the presence and quantity of anti-BSEP antibodies in the biological sample of the subject according to the method of claim 1;
comparing the quantity of anti-BSEP antibodies from the biological sample to a quantity of anti-BSEP antibodies in a control sample or to a previously measured quantity of anti-BSEP antibodies from another biological sample of the subject; and
determining whether the subject has BSEP deficiency or is likely to develop BSEP deficiency from the comparison.

12. A method of treating a subject having, or likely to have, a BSEP deficiency, the method comprising:
detecting the presence and quantity of anti-BSEP antibodies in the biological sample of the subject according to the method of claim 1;
determining whether the subject has BSEP deficiency or is likely to develop BSEP deficiency from the detecting; and
if the subject is determined to have BSEP deficiency, administer a treatment selected from the group consisting of immunosuppressive therapy, plasmapheresis, immunoadsorption therapy, rituximab, or a combination thereof.

13. A kit for detecting the presence and quantity of anti-BSEP antibodies, the kit comprising
a sample receiving region; and
a surface comprising a test region comprising one or more immobilized BSEP peptides.

14. The kit according to claim 13, wherein the sample receiving region, the surface, and test region are configured for enzyme-linked immunosorbent assay (ELISA).

15. The kit according to claim 13, wherein the one or more immobilized BSEP peptides comprises one or more of a full length BSEP peptide (SEQ ID NO:1), or one or more epitopes of BSEP.

16. The kit of claim 15, wherein the one or more epitopes of BSEP are selected from the group consisting of the peptide of SEQ ID NO: 2, one or more epitopes of the full length BSEP peptide having a length of 18-20 amino acids, and combinations thereof.

17. The kit of claim 15, wherein the one or more epitopes are selected from the group consisting of the peptide of SEQ ID NO: 2, the peptide of SEQ ID NO: 3, the peptide of SEQ ID NO: 4, the peptide of SEQ ID NO: 5, the peptide of SEQ ID NO: 6, the peptide of SEQ ID NO: 7, the peptide of SEQ ID NO: 8, and combinations thereof.

18. The kit of claim 13, wherein the one or more immobilized BSEP peptides is a single BSEP peptide selected from the group consisting of the full length BSEP peptide (SEQ ID NO: 1), the peptide of SEQ ID NO: 2, the peptide of SEQ ID NO: 3, the peptide of SEQ ID NO: 4, the peptide of SEQ ID NO: 5, the peptide of SEQ ID NO: 6, the peptide of SEQ ID NO: 7, or the peptide of SEQ ID NO: 8.

19. The kit of claim 13, wherein the one or more immobilized BSEP peptides comprises a panel of 2 or more peptides.

20. The kit of claim 19, wherein the panel of 2 or more immobilized BSEP peptides comprises one of:
the full length BSEP peptide (SEQ ID NO: 1), and the peptide of SEQ ID NO: 2;
the full length BSEP protein (SEQ ID NO:1), and any one or more of the peptide of SEQ ID NO: 2, the peptide of SEQ ID NO: 3, the peptide of SEQ ID NO: 4, the peptide of SEQ ID NO: 5, the peptide of SEQ ID NO: 6, the peptide of SEQ ID NO: 7, the peptide of SEQ ID NO: 8, or a combination thereof;
the peptide of SEQ ID NO: 2), and any one or more of the peptide of SEQ ID NO: 2, the peptide of SEQ ID NO: 3, the peptide of SEQ ID NO: 4, the peptide of SEQ ID NO: 5, the peptide of SEQ ID NO: 6, the peptide of SEQ ID NO: 7, the peptide of SEQ ID NO: 8, or a combination thereof;
any 2 or more of the peptide of SEQ ID NO: 2, the peptide of SEQ ID NO: 3, the peptide of SEQ ID NO: 4, the peptide of SEQ ID NO: 5, the peptide of SEQ ID NO: 6, the peptide of SEQ ID NO: 7, or the peptide of SEQ ID NO: 8, or a combination of thereof; or
the full length BSEP protein (SEQ ID NO:1), the peptide of SEQ ID NO: 2, and any one or more of the peptide of SEQ ID NO: 2, the peptide of SEQ ID NO: 3, the peptide of SEQ ID NO: 4, the peptide of SEQ ID NO: 5, the peptide of SEQ ID NO: 6, the peptide of SEQ ID NO: 7, the peptide of SEQ ID NO: 8, or a combination thereof.


SEQUENCE LISTING (8 sequences)

SEQ ID NO: 1 - protein, 1321 aa, synthetic construct
MSDSVILRSIKKFGEENDGFESDKSYNNDKKSRLQDEKKGDGVRVGFFQLFRFSSSTDIWLMFVGSLCAFLHGIAQPGVLLIFGTMTDVFIDYDVELQELQIPGKACVNNTIVWTNSSLNQNMTNGTRCGLLNIESEMIKFASYYAGIAVAVLITGYIQICFWVIAAARQIQKMRKFYFRRIMRMEIGWFDCNSVGELNTRFSDDINKINDAIADQMALFIQRMTSTICGFLLGFFRGWKLTLVIISVSPLIGIGAATIGLSVSKFTDYELKAYAKAGVVADEVISSMRTVAAFGGEKREVERYEKNLVFAQRWGIRKGIVMGFFTGFVWCLIFLCYALAFWYGSTLVLDEGEYTPGTLVQIFLSVIVGALNLGNASPCLEAFATGRAAATSIFETIDRKPIIDCMSEDGYKLDRIKGEIEFHNVTFHYPSRPEVKILNDLNMVIKPGEMTALVGPSGAGKSTALQLIQRFYDPCEGMVTVDGHDIRSLNIQWLRDQIGIVEQEPVLFSTTIAENIRYGREDATMEDIVQAAKEANAYNFIMDLPQQFDTLVGEGGGQMSGGQKQRVAIARALIRNPKILLLDMATSALDNESEAMVQEVLSKIQHGHTIISVAHRLSTVRAADTIIGFEHGTAVERGTHEELLERKGVYFTLVTLQSQGNQALNEEDIKDATEDDMLARTFSRGSYQDSLRASIRQRSKSQLSYLVHEPPLAVVDHKSTYEEDRKDKDIPVQEEVEPAPVRRILKFSAPEWPYMLVGSVGAAVNGTVTPLYAFLFSQILGTFSIPDKEEQRSQINGVCLLFVAMGCVSLFTQFLQGYAFAKSGELLTKRLRKFGFRAMLGQDIAWFDDLRNSPGALTTRLATDASQVQGAAGSQIGMIVNSFTNVTVAMIIAFSFSWKLSLVILCFFPFLALSGATQTRMLTGFASRDKQALEMVGQITNEALSNIRTVAGIGKERRFIEALETELEKPFKTAIQKANIYGFCFAFAQCIMFIANSASYRYGGYLISNEGLHFSYVFRVISAVVLSATALGRAFSYTPSYAKAKISAARFFQLLDRQPPISVYNTAGEKWDNFQGKIDFVDCKFTYPSRPDSQVLNGLSVSISPGQTLAFVGSSGCGKSTSIQLLERFYDPDQGKVMIDGHDSKKVNVQFLRSNIGIVSQEPVLFACSIMDNIKYGDNTKEIPMERVIAAAKQAQLHDFVMSLPEKYETNVGSQGSQLSRGEKQRIAIARAIVRDPKILLLDEATSALDTESEKTVQVALDKAREGRTCIVIAHRLSTIQNADIIAVMAQGVVIEKGTHEELMAQKGAYYKLVTTGSPIS

SEQ ID NO: 2 - protein, 131 aa, synthetic construct
RLSTVRAADTIIGFEHGTAVERGTHEELLERKGVYFTLVTLQSQGNQALNEEDIKDATEDDMLARTFSRGSYQDSLRASIRQRSKSQLSYLVHEPPLAVVDHKSTYEEDRKDKDIPVQEEVEPAPVRRILK

SEQ ID NO: 3 - protein, 20 aa, synthetic construct
DSVILRSIKKFGEENDGFES

SEQ ID NO: 4 - protein, 20 aa, synthetic construct
IAAARQIQKMRKFYFRRIMR

SEQ ID NO: 5 - protein, 20 aa, synthetic construct
EANAYNFIMDLPQQFDTLVG

SEQ ID NO: 6 - protein, 20 aa, synthetic construct
QAQLHDFVMSLPEKYETNVG

SEQ ID NO: 7 - protein, 20 aa, synthetic construct
SQGSQLSRGEKQRIAIARAI

SEQ ID NO: 8 - protein, 20 aa, synthetic construct
ELMAQKGAYYKLVTTGSPIS